(19) United States
(12) Patent Application Publication
(10) Pub. No.: US 2020/0008923 A1
(43) Pub. Date: Jan. 9, 2020

(54) IMPLANT DELIVERY SYSTEM AND METHOD

(71) Applicant: SIENTRA, INC., Santa Barbara, CA (US)

(72) Inventors: Steven Charles GEIGER, Prescott, AZ (US); Dan Alan KRIEGER-CARLISLE, Santa Barbara, CA (US); Douglas M. GOODNER, Goleta, CA (US); Oliver Christian BENNETT, Jersey City, NJ (US); Nancy WERNER, Santa Barbara, CA (US)

(21) Appl. No.: 16/506,759

(22) Filed: Jul. 9, 2019

Related U.S. Application Data
(60) Provisional application No. 62/695,540, filed on Jul. 9, 2018.

(51) Int. Cl. A61F 2/00

(52) U.S. Cl. CPC A61F 2/0095; A61F 2/12

(57) ABSTRACT

An implant delivery device includes a first closed sidewall, a second closed sidewall, a third closed sidewall, and an open end. The second closed sidewall is disposed opposite the first closed sidewall. The third closed sidewall is disposed between the first closed sidewall and the second closed sidewall. The open end is disposed opposite the third closed sidewall and disposed between the first closed sidewall and the second closed sidewall.

### CROSS-REFERENCE TO RELATED APPLICATIONS

This application claims priority to U.S. Provisional Application No. 62/695,540, filed Jul. 9, 2018, the contents of which are incorporated herein by reference in their entirety.

### BACKGROUND

Implant delivery devices have multiple orifices, or a slot or seam, one through which the implant is placed into the delivery device and one through which the implant is then inserted into the patient. Multiple orifices and/or other slots or seams complicate the device and procedure and increase the chance of handling, loading, and implant delivery errors.

The physical handling of implants is also believed to increase infection risk to the patient, and therefore devices that reduce or even eliminate handling of the implant prior to delivery would be preferred to reduce the risk of patient injury.

A simpler solution with a single opening, that reduces or eliminates the physical handling of implants prior to insertion, and that is easier to use, would be desirable.

### BRIEF SUMMARY

According to an aspect, an implant delivery device includes a body portion and an implant delivery portion. The body portion includes a first sidewall, a second sidewall, and a closed end. The implant delivery portion is disposed opposite to the closed end. The implant delivery portions include a first sidewall, a second sidewall, and an orifice. The first sidewall is angled with respect to the first sidewall of the body portion, and the second sidewall is angled with respect to the second sidewall of the body portion.

The closed end may include an end wall bonded to at least one face of the body portion.

The closed end may include a bond between at least one face of the body portion.

The body portion may include a lay flat tube. The thermal bond may include a bond closing the lay flat tube.

The implant delivery portion may be configured to invert such that the orifice is disposed inside the body portion.

The implant delivery portion may be configured to receive an implant while in an inverted configuration such that the implant is disposed inside the body portion.

The implant delivery portion may be configured to return to a not inverted configuration while an implant is disposed in the body portion. In the not inverted configuration, the orifice may not be disposed inside the body portion.

An interior surface of the implant delivery portion may include a friction reducing coating.

The friction reducing coating may be hydrophilic.

An interior surface of the implant delivery portion may include an antimicrobial treatment.

The orifice may be an only opening of the implant delivery device.

According to another aspect, an implant delivery device includes a first closed sidewall, a second closed sidewall, a third closed sidewall, and an open end. The second closed sidewall is disposed opposite the first closed sidewall. The third closed sidewall is disposed between the first closed sidewall and the second closed sidewall. The open end is disposed opposite the third closed sidewall and disposed between the first closed sidewall and the second closed sidewall. A face of the implant delivery device disposed between the first closed sidewall and the second closed sidewall is configured to fold such that a cross-sectional area of the open end is reduced.

The face of the implant delivery device may include an indicia. The indicia may extend from proximal a corner of the first closed sidewall and the open end toward the second closed sidewall.

The indicia may include a crease.

The face may include a protruding tab.

The indicia may extend at approximately a 45 degree angle with respect to the open end.

The device may include a second indicia extending from the first indicia towards the open end.

According to another aspect, a method of manufacturing an implant delivery device includes: providing a section of lay flat tubing; inverting the lay flat tubing to arrange the lay flat tubing in an inverted configuration; applying a friction reducing treatment to the inverted lay flat tubing; inverting the lay flat tubing to arrange the lay flat tubing in a not inverted configuration; and sealing an end of the section of lay flat tubing.

The lay flat tubing may include a thermoplastic material, and sealing the end of the section of lay flat tubing includes thermally bonding the thermoplastic material.

In a not inverted configuration, an interior surface of the lay flat tubing may be textured.

The applying the friction reducing treatment may include dipping the inverted lay flat tubing in a lubricious material.

The method may include curing the lubricious material before the inverting the lay flat tubing to arrange the lay flat tubing in a not inverted configuration.

The lubricious material may be hydrophilic.

According to another aspect, a method of inserting an implant includes: providing an implant delivery device, the device including: a first closed sidewall, a second closed sidewall disposed opposite the first closed sidewall, a third closed sidewall disposed between the first closed sidewall and the second closed sidewall, and an open end disposed opposite the third closed sidewall and disposed between the first closed sidewall and the second closed sidewall; inserting the implant into the implant delivery device through the open end; folding the first closed sidewall towards the second closed sidewall to reduce a cross sectional area of an orifice provided by the open end; and applying a force to the implant delivery device to cause the implant to move towards and through the orifice.

The method may include folding the second closed sidewall towards the first closed sidewall to reduce a cross sectional area of the orifice.

The method may include removing a corner formed by the open end and the second closed side wall.

The removing may include cutting.

An interior surface of the implant delivery device may include a friction reducing treatment.

The friction reducing treatment may include a hydrophilic coating.

The method may include hydrating the friction reducing treatment.

### BRIEF DESCRIPTION OF THE DRAWINGS

For a better understanding of the aforementioned embodiments as well as additional embodiments thereof, reference should be made to the Detailed Description below, in conjunction with the following drawings in which like reference numerals refer to corresponding parts throughout the figures.

FIG. 1 is a perspective view of an implant delivery device according to an embodiment.

FIG. 2A is a front view of the implant delivery device of FIG. 1.

FIG. 2B is a side view of the implant delivery device of FIG. 1.

FIG. 2C is a bottom view of the implant delivery device of FIG. 1.

FIG. 3A is a profile view of an outline of a shape of an exemplary implant delivery device.

FIG. 3B is a profile view of an outline of a shape of an exemplary implant delivery device.

FIG. 3C is a profile view of an outline of a shape of an exemplary implant delivery device.

FIG. 3D is a profile view of an outline of a shape of an exemplary implant delivery device.

FIG. 3E is a profile view of an outline of a shape of an exemplary implant delivery device.

FIG. 3F is a profile view of an outline of a shape of an exemplary implant delivery device.

FIG. 3G is a profile view of an outline of a shape of an exemplary implant delivery device and a functional diagram illustrating an exemplary operation of the implant delivery device.

FIG. 3H is a profile view of an outline of a shape of an exemplary implant delivery device.

FIG. 3I is a profile view of an outline of a shape of an exemplary implant delivery device.

FIG. 4 is a perspective view of an implant delivery device according to an embodiment.

FIG. 5A is a front view of the implant delivery device of FIG. 4.

FIG. 5B is a side view of the implant delivery device of FIG. 4.

FIG. 5C is a bottom view of the implant delivery device of FIG. 4.

FIG. 6 is a perspective view of an implant delivery device according to an embodiment.

FIG. 7A is a front view of the implant delivery device of FIG. 6.

FIG. 7B is a side view of the implant delivery device of FIG. 6.

FIG. 7C is a bottom view of the implant delivery device of FIG. 6

FIG. 8A is a front view of an implant delivery device prior to implant loading according to an embodiment.

FIG. 8B is a front view of an implant delivery device with an implant partially inserted according to an embodiment.

FIG. 8C is a front view of an implant delivery device with an implant loaded according to an embodiment.

FIG. 8D is a front view of an implant delivery device with an implant loaded and delivery portion extended according an embodiment.

FIG. 8E is a front view of an implant delivery device with delivery portion extended and the implant disposed in the delivery portion according to an embodiment.

FIG. 8F is a front view of an implant delivery device with delivery portion extended and the implant partially extending from the delivery portion according to an embodiment.

FIG. 9 is a perspective cross-sectional view of an implant delivery device according an embodiment.

FIG. 10A illustrates a front view of an implant delivery device in an unfolded configuration according to an embodiment.

FIG. 10B illustrates a bottom view of the implant delivery device of FIG. 10A in an open configuration.

FIG. 10C illustrates a side view of the implant delivery device of FIG. 10A loaded with an implant.

FIG. 10D illustrates a front view of the implant delivery device of FIG. 10A loaded with an implant in a one side folded configuration.

FIG. 10E illustrates a front view of the implant delivery device of FIG. 10A loaded with an implant in a folded configuration.

FIG. 10F illustrates a front view of the implant delivery device of FIG. 10A loaded with an implant in a folded configuration schematically showing a closure.

FIG. 10G illustrates a front view of the implant delivery device of FIG. 10A loaded with an implant in a folded configuration.

FIG. 11A illustrates a front view of an implant delivery device according to an embodiment.

FIG. 11B illustrates a front view of the implant delivery device of FIG. 11A in a folded configuration.

FIG. 11C illustrates a front view of the implant delivery device of FIG. 11A in a delivery configuration.

FIG. 12 illustrates a process view of manufacturing an implant delivery device.

### DETAILED DESCRIPTION

Reference will now be made in detail to embodiments, examples of which are illustrated in the accompanying drawings and figures. In the following detailed description, numerous specific details are set forth in order to provide a thorough understanding of the invention. However, it will be apparent to one of ordinary skill in the art that the invention may be practiced without these specific details. In other instances, well-known methods, procedures, components, circuits and networks have not been described in detail so as not to unnecessarily obscure aspects of the embodiments.

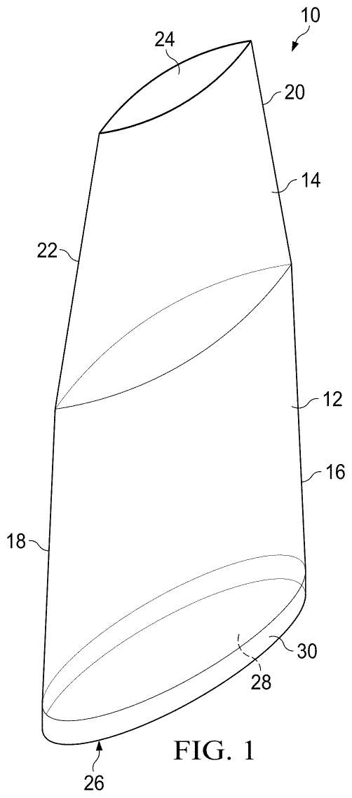

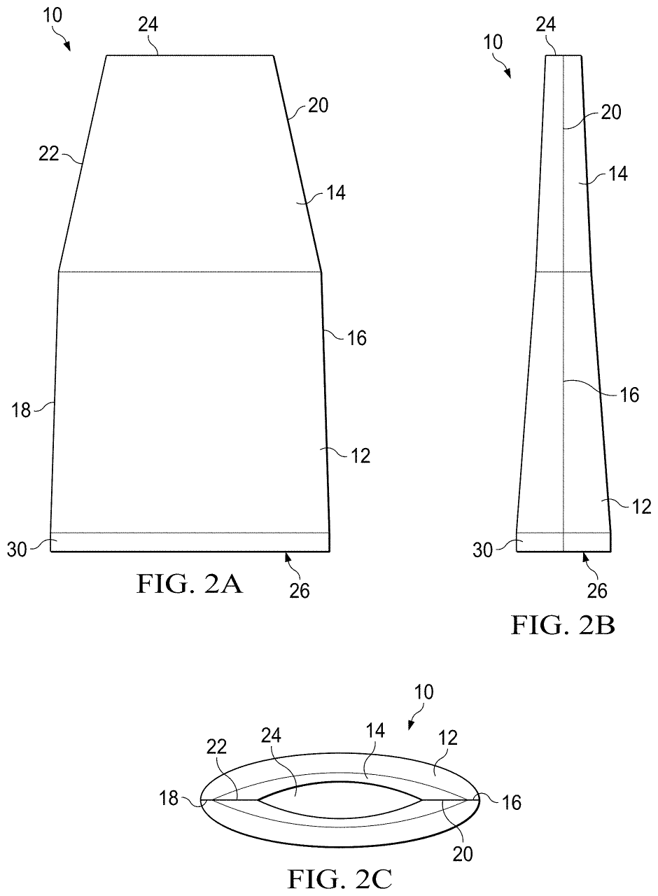

FIG. 1 is a perspective view of an implant delivery device according to an embodiment. FIG. 2A is a front view of the implant delivery device of FIG. 1. FIG. 2B is a side view of the implant delivery device of FIG. 1. FIG. 2C is a bottom view of the implant delivery device of FIG. 1. In some embodiments, the implant delivery device is a breast implant or other implant insertion device. The implant delivery device may be used to insert the implant without touching the implant thereby reducing the risk of infection or damage to the implant. With reference to FIGS. 1 and 2, the implant delivery device 10 includes a body portion 12 and a delivery portion 14. Side walls 16 and 18 of the body portion 12 may be straight. Side walls 20 and 22 of the delivery portion may be angled with respect to the side walls 16 and 18. The angle of the side walls 16 and 18 provides for a width of the orifice 24 smaller than a width of the body portion 12. Preferably, the width of the orifice 24 is smaller than a size of the implant so that the implant remains inside the device 10 in a resting state when force is not applied to the device 10, and a width of the body portion 12 is larger than the size of the implant to accommodate the implant therein.

The implant delivery device 10 includes a single orifice 24 at one end (e.g., at the end of the sidewalls 20 and 22, and a closed end 26 at the opposite end of the implant delivery device 10. An end wall 28 may be bonded to the body portion 12 by a thermal weld, glue, tape, or other securement at the bonding region 30. In some embodiments, all sides of the implant delivery device 10 may be sealed. An exemplary advantage of such an embodiment is the maintenance of sterile conditions inside the implant delivery device. The surgeon may cut the implant delivery device to form the orifice.

The body portion 12 of the device 10 may be generally cylindrical with a single orifice 24 at one end of the delivery portion 14 having a reduced width relative to the body portion 12. The single orifice 24 can be used for placing the implant into the insertion device 10 and for delivering the implant into the patient. The closed end 26 of the implant delivery device may prevent the implant from slipping out of the insertion device 10 prior to insertion into the patient, limit or avoid contamination from entering the device 10 and reaching the implant, and house the implant while lubrication (e.g., saline or betadine) is applied to the implant or the inner surface of the implant delivery device. An exemplary advantage of the single opening is that it holds the contents of the lubrication (or other hydration) making the application of the lubrication or hydration easier, less messy, use a smaller quantity of lubrication or hydration and be more effective.

In some embodiments, the region 30 includes a flexible elastomer in place of the closed end 26. The flexible elastomer may be stretched to allow the implant to fit through and then return to its original size. In this manner, the implant can be securely loaded into the implant delivery device in cases where the orifice 24 is smaller than the implant.

In some embodiments, the implant delivery device 10 is pre-loaded with an implant for insertion into the patient, for example, in a manufacturing clean room to provide the least amount of contamination possible. The implant delivery device 10 and pre-loaded implant can be packaged in sterile packaging in preparation for delivery to the patient. For use, the implant delivery device 10 and pre-inserted implant can be removed from sterile packaging, and, in some embodiments, the lubricious coating can be hydrated if required. By being pre-loaded in the implant delivery device (e.g., by the manufacturer in some embodiments), the implant may be provided to the surgeon ready for delivery to the patient in a sterile state. In such an embodiment, the implant delivery device 10 with the implant disposed in the body 12 may together be disposed in a box or packaging such as shipping packaging that protects the combined device 10 and implant for transit.

The incision site may be slightly smaller than a width of the orifice 24. The orifice 24 may be inserted slightly into the site such that the side walls 20 and 22 extend into the surgical opening (e.g., 1 cm). When the implant is then deployed into the surgical site, as discussed in more detail below, the delivery may be touchless as the surgeon will not have directly touched the implant nor will the implant have come into contact with any external surface of the patient or environment.

In an embodiment, the implant is a breast implant, such as a silicone gel type implant (which may be an elastomer silicone shell enclosing a silicone gel). Other implant types, such as a saline implant, other composite filler type implants or tissue expander can also be used with the implant delivery device 10.

The implant can be placed inside the implant delivery device 10 through the single orifice 24 of the implant delivery device. The orifice 24 and other device dimensions can be sized according to the intended implant such that the end use is not required to make adjustments or modifications such as cuts, stretching, or additions or deletions.

In some embodiments, the implant delivery device is constructed of a material and structure that can be sterilized and/or is sterilizable. Sterilization methods include, but are not limited to, ethylene oxide sterilization, chlorine dioxide sterilization, hydrogen peroxide sterilization, gamma ray sterilization, electron beam sterilization, wet heat sterilization, dry heat sterilization, or a combination thereof. In embodiments where the implant is pre-loaded with the implant delivery device, the implant delivery device may be made of materials that can be usable at least once after being subjected to the sterilization method applied to the implant.

In some embodiments, at least a portion of the implant delivery device 10 includes a material that is at least partially transparent or translucent. The transparent or semi-transparent portions of the implant delivery device 10 may allow a surgeon to view the position of an enclosed implant and/or the position or motion of the implant during manipulation. This may be particularly advantageous in the case of a shaped implant to facilitate the delivery of the implant in the desired orientation. For example, at least a portion of the implant delivery device 10 may include transparent or translucent polymer film to allow a surgeon to view at least a portion of the implant.

In some embodiments, the implant delivery device 10 may be constructed of a flexible or semi flexible polymer or copolymer material. For example, the implant delivery device 10 may include one or more of polyethylene, polypropylene, polytetrafluoroethylene, polycarbonate, polyethylene terephthalate, fluoropolymer, ethylene vinyl acetate, polyvinylchloride, polyvinylchloride and silicon. The material may be provided as a film and bonded along a seam to provide the generically cylindrical shape of the body portion 12. The material may also be provided by a lay flat tube that may limit or avoid the need for seams. An advantage of a lay flat tube is that manufacturing steps for bonding a vertical seam may be limited or avoided thereby increasing yields and decreasing manufacturing costs. The lay flat tube may also limit or reduce the number of seams thereby reducing possible weak points and increasing the strength of the device 10. Preferably, the device 10 will not fail or yield under forces that may be exerted on the device by a surgeon.

In some embodiments, one or more of the internal surfaces of the implant delivery device 10 may be fabricated or treated to provide a low friction coefficient. This may be achieved through the use of a lubricious coating. A lubricious coating or surface may include a coating or surface of an object having slipperiness, low friction and/or smoothness. This description is exemplary in nature and a lubricious coating or surface is not defined by or limited thereto. For example, in some embodiments, at least a portion of at least one or more of the internal surfaces of the implant delivery device 10 can be lubricious. The lubricity may be achieved with or without a coating on the material of the implant delivery device 10. The lubricity may be provided or enhanced by a hydrophilic coating or a hydrophobic coating. In some embodiments, the lubricity includes a combination of the lubricious nature of the material (for example forming the walls) of the implant delivery device 10, and a coating on the material of the implant delivery device 10.

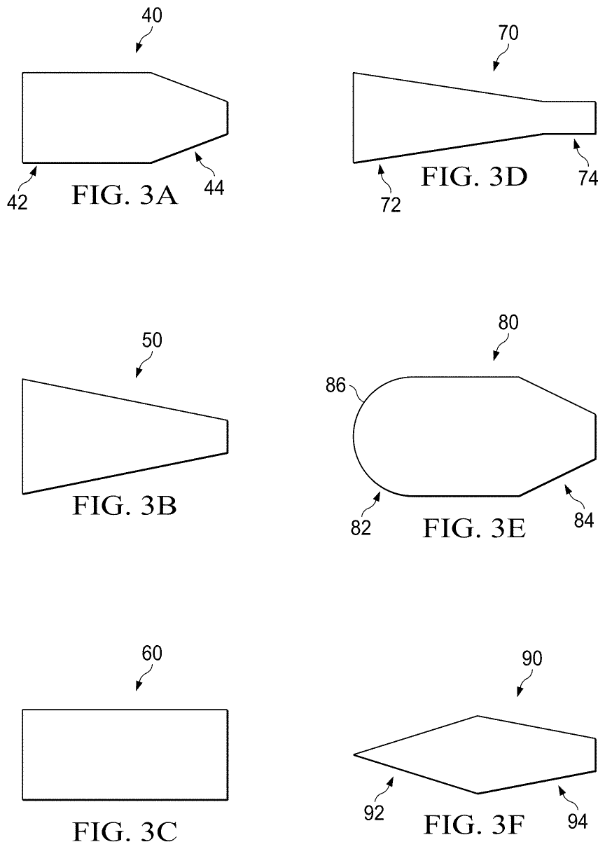

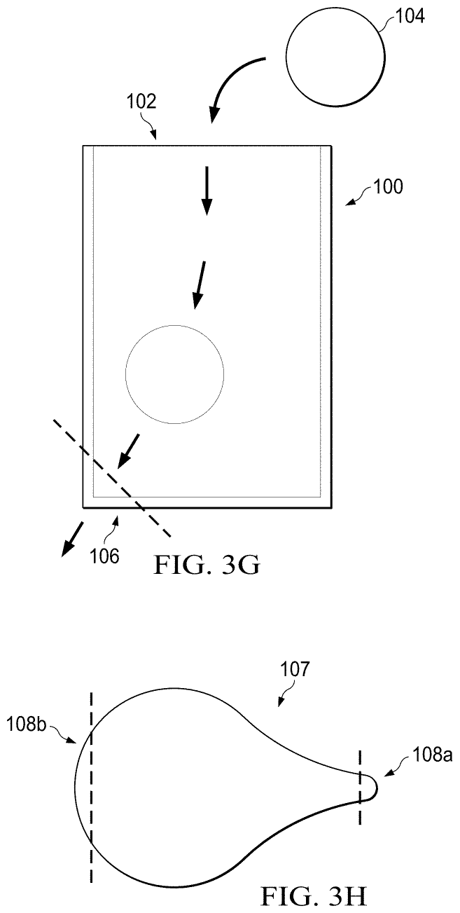

The size and/or shape of the implant delivery device 10 may be different based on surgeon requirements and/or patient requirements. For example, the single orifice 24 may have different sizes and dimensions, including angled or straight sides. The body portion 12 for holding the implant can also have different sizes and dimensions, including flat, curved or other shaped sealed ends. Some non-limiting embodiments are shown in FIGS. 3A-3I, illustrating shape outlines for embodiments of implant delivery devices.

FIG. 3A illustrates an insertion device 40 having a body portion 42 with straight walls and a delivery portion 44 with angled walls, similar to the embodiment discussed above with respect to FIGS. 1 and 2.

FIG. 3B illustrates an insertion device 50 having a body portion and a delivery portion with angled walls. Forming the body portion combined with the delivery portion may provide advantages in the manufacturing process of simpler or fewer seams and less wasted material.

FIG. 3C illustrates an insertion device 60 having a body portion and a delivery portion with straight walls. Forming the body portion combined with the delivery portion may provide advantages in the manufacturing process of simpler or fewer seams and less wasted material. This embodiment may be particularly advantageous with the device is constructed from lay flat tubing stock as only one seam at the end of the device may be needed.

FIG. 3D illustrates an insertion device 70 having a body portion 72 with angled walls and a delivery portion 74 with straight walls. This embodiment may be particularly advantageous when the implant is to be delivered farther into the surgical site. The straight walls of the delivery portion facilitates easy insertion of the insertion device into the surgical site.

FIG. 3E illustrates an insertion device 80 having a body portion 82 with straight walls and a delivery portion 84 with angled walls. An end portion 86 of the body portion 82 has a rounded shape. This embodiment may be particularly advantageous with large implants or pre-packaged implants as the rounded shape provides a location to more easily accommodate the implant.

FIG. 3F illustrates an insertion device 90 having a body portion 92 with angled walls and a delivery portion 94 with angled walls. The walls of the body portion 92 are angled in the opposite direction compared to the angled walls of the delivery portion 94. This embodiment may be particularly advantageous as the angled walls of the body portion may facilitate moving the implant towards and through the delivery portion 94.

In the non-limiting embodiments show in FIGS. 3A-3F, the single orifice may be used for placing the implant inside the insertion device and for delivering the implant into the patient. Alternatively, as shown in FIG. 3G, an insertion device 100 may include a single orifice 102 for loading of an implant 104, and a cut-away or removable portion 106 that can be removed (e.g., by cutting, slicing, tearing, or selectively separating) to provide for implant delivery. In some embodiments, the insertion device can include a guide, pre-cut or weakened region that can be used to selectively remove a portion of the insertion device.

FIG. 3H Illustrates an insertion device 107 having a teardrop shape. The implant device may be sealed around an entire periphery. A surgeon may select the size of the opening for an orifice by making a cut on the device. For a small orifice, the surgeon may cut proximal to location 108a. For a larger orifice, the surgeon may cut proximal to location 108b.

FIG. 3I illustrates an insertion device 109 having an hourglass shape. The ends 111a and 111b may be open. The hour glass shape includes a narrow portion proximal to the end 111b. The ends 111a and 111b may be substantially the same size. An exemplary advantage of this embodiment is that the implant does not proceed to the insertion end until an encouraging force is applied by the surgeon. The insertion end may remain flatter facilitating positioning of the insertion end.

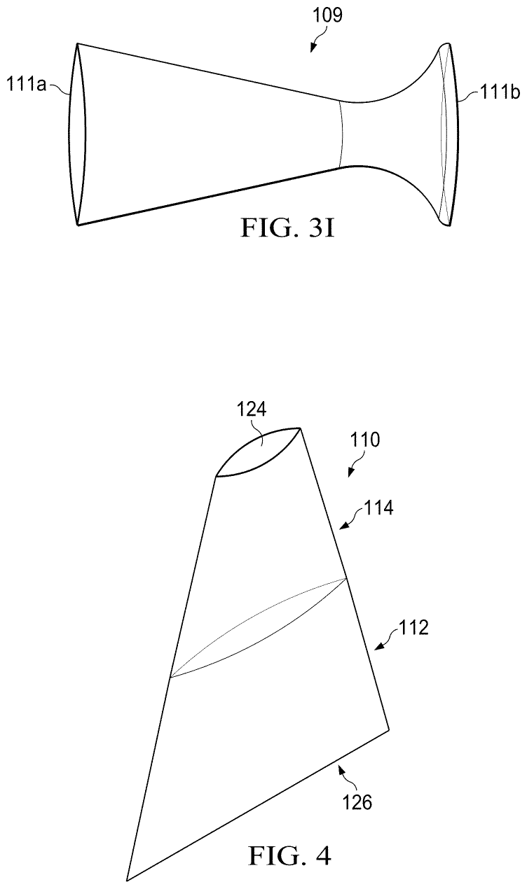

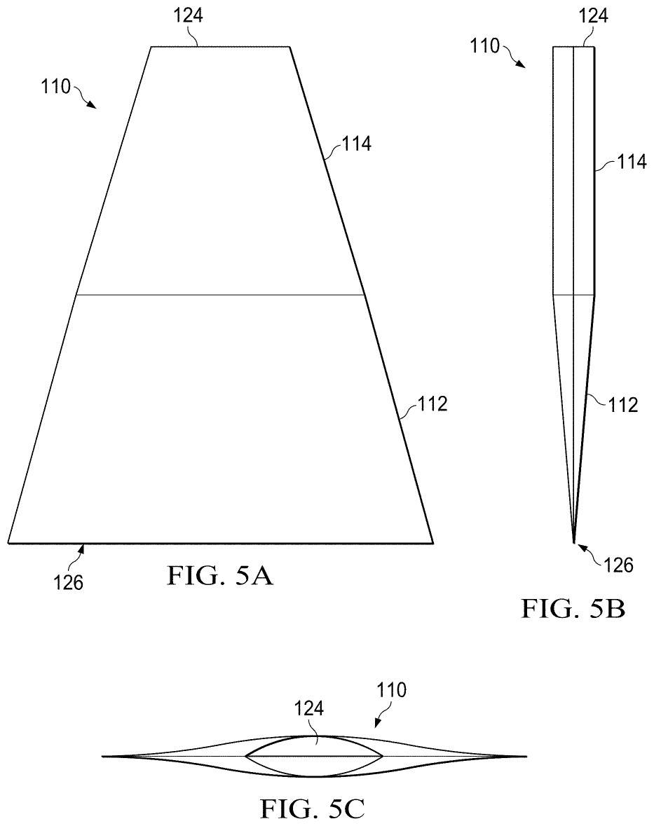

FIG. 4 is a perspective view of an implant delivery device according to an embodiment. FIG. 5A is a front view of the implant delivery device of FIG. 4. FIG. 5B is a side view of the implant delivery device of FIG. 4. FIG. 5C is a bottom view of the implant delivery device of FIG. 4. With reference to FIGS. 4 and 5, an implant delivery device 110 includes a body portion 112 and a delivery portion 114. The implant delivery device includes a single orifice 124. While illustrated with angled side walls for both the body portion 112 and delivery portion 114, it will also be appreciated that the body portion 112 may include straight walls. At an end opposite to the orifice 124, the body portion 112 includes a sealed end 126. The sealed end 126 may be formed by a thermal weld, glue, tape, or other securement. An exemplary advantage of providing a sealed end 126 is that the manufacturing complexity of including an end wall (such as the end wall 28 of FIG. 1) can be avoided.

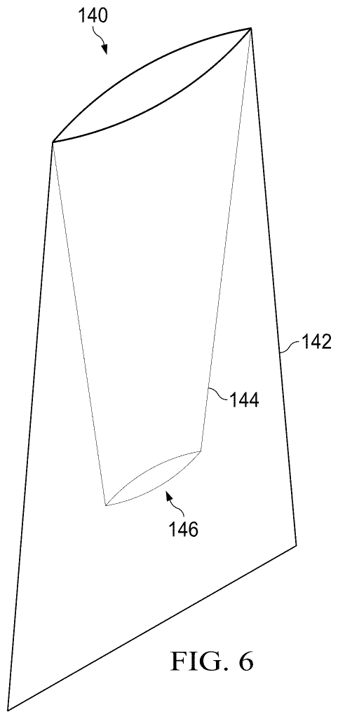

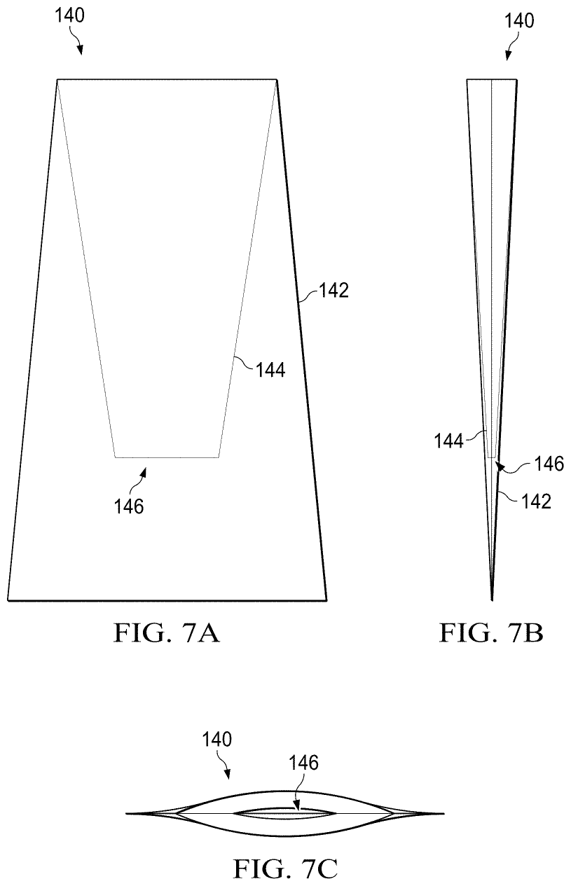

FIG. 6 is a perspective view of an implant delivery device according to an embodiment. FIG. 7A is a front view of the implant delivery device of FIG. 6. FIG. 7B is a side view of the implant delivery device of FIG. 6. FIG. 7C is a bottom view of the implant delivery device of FIG. 6. With reference to FIGS. 6 and 7, an implant delivery device 140 includes a body portion 142 and a delivery portion 144. The implant delivery device includes a single orifice 146. While illustrated with angled side walls for both the body portion 142 and delivery portion 144, it will also be appreciated that the body portion 142 may include straight walls. At an end opposite to the orifice 146, the body portion 112 includes a sealed end.

FIGS. 6 and 7 illustrate the implant delivery device 140 in a configuration in which the delivery portion 144 is inverted. In this configuration, the orifice 146 is disposed inside the body portion 142. Preferably a length of the delivery portion 144 is less than a length of the body portion 142 by a margin of approximately equal to or greater than a width of the implant.

The implant may be loaded into the sealed-end chamber provided by the body portion 142 by being passed through the inverted delivery portion 144 and through the orifice 146 by the surgeon. The implant delivery device 140 can then be turned such that gravity pulls the implant towards the orifice 142 causing the inverted delivery portion 144 revert to its not inverted configuration. The delivery portion 144 may also be manually manipulated to the not inverted configuration.

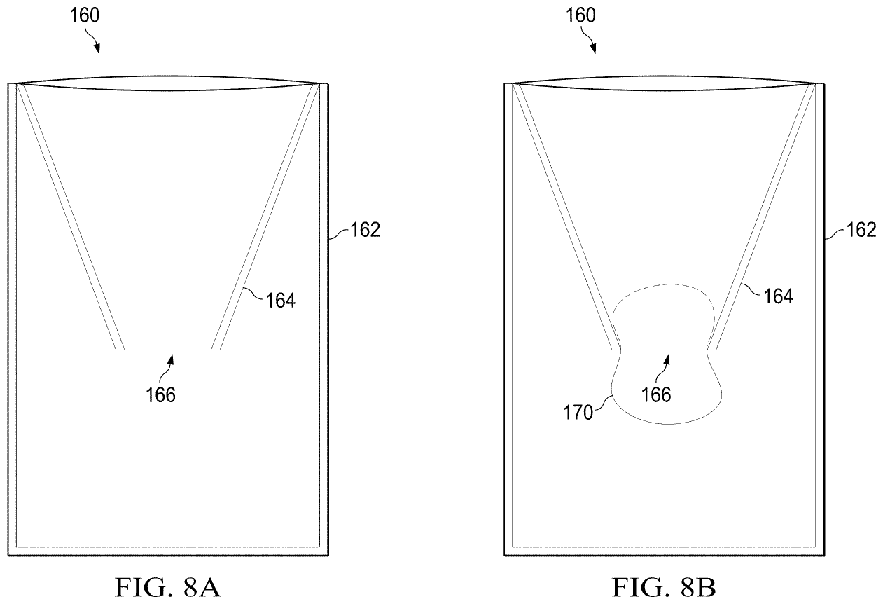

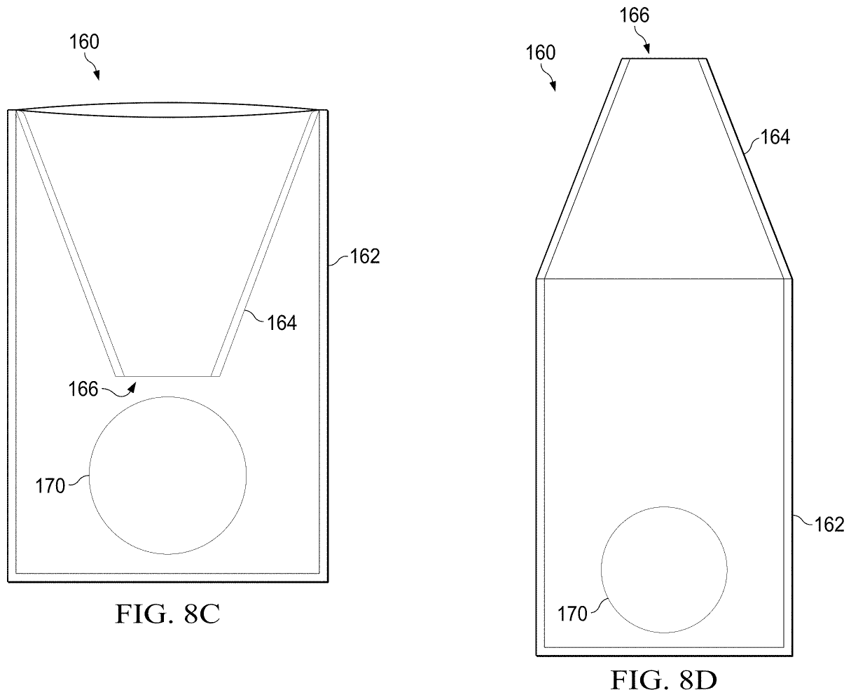

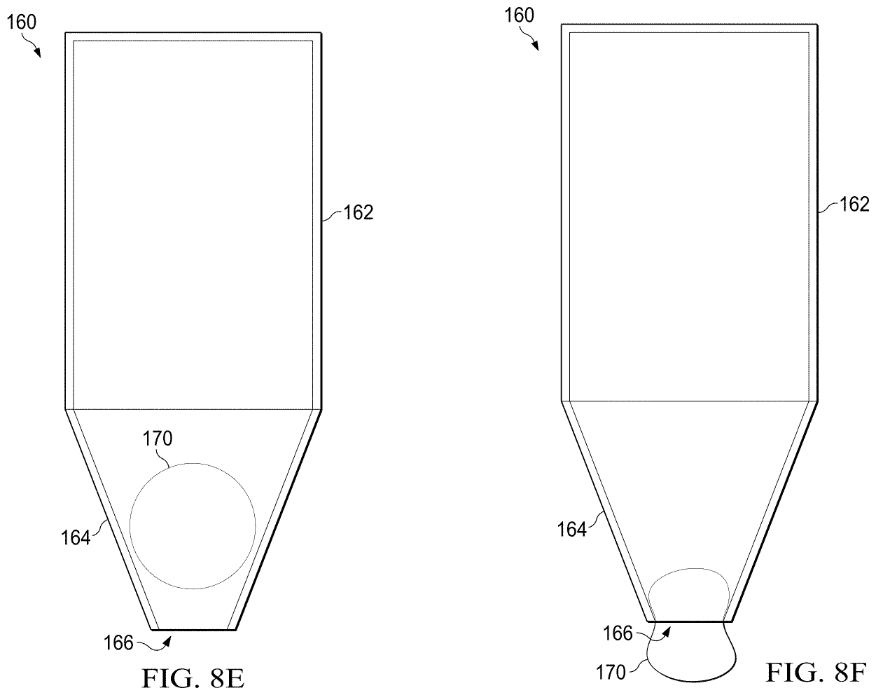

With reference to FIGS. 8, FIG. 8A is a front view of an implant delivery device 160 (such as any of the implant delivery devices discussed above) prior to implant loading. The implant delivery device 160 is in a configuration in which the delivery portion 164 is inverted such that the orifice 166 is disposed in the body portion 162. FIG. 8B is a front view of the implant delivery device 160 with an implant 170 inserted into the orifice 166. FIG. 8C is a front view of an implant delivery device 160 with an implant loaded and disposed in the body portion 162. FIG. 8D is a front view of an implant delivery device 160 with an implant loaded and disposed in the body portion 162.

At this stage, the orifice 166 is inserted into the surgical site. Preferably, the incision in the surgical site is slightly smaller than a width of the orifice 166 such that orifice 166 can be securely placed into the surgical site to limit exposure of the implant 170 to the exterior environment of the surface of the skin, which may have bacteria and other undesirable contamination. Flexibility of the implant and/or the material of the implant delivery device 160 allows the implant to pass through the orifice 166 with the application of a gentle force as described next. It will be appreciated that the implant delivery device 166 may also be used to deliver the implant to the patient without inserting the orifice 166 into the surgical site.

FIG. 8E is a front view of the implant delivery device 160 with delivery portion 164 extended and primed for implant delivery. The implant 170 may be positioned within the delivery portion 164 by orienting the insertion device 160 to allow gravity to move the implant 170. The surgeon may also encourage the implant 170 to move into the delivery portion 164 by applying gentle force from behind the implant (e.g., from the side of the sealed end). Preferably, the surgeon does not apply force directly to the implant 170 to limit potential damage to the implant. For example, the surgeon may squeeze the sidewalls of the delivery device 160 near the sealed end and make a motion towards the delivery portion 164. This motion may encourage the movement of the implant without applying force directly to the implant.

FIG. 8F is a front view of the implant delivery device 160 with the delivery portion 164 extended and the implant 170 partially extending from the orifice 166. The surgeon may continue the motion described above (e.g., squeezing the sidewalls of the delivery portion 164 together from behind the implant 170 thereby encouraging the implant 170 to move towards and through the orifice 166 and into the surgical site.

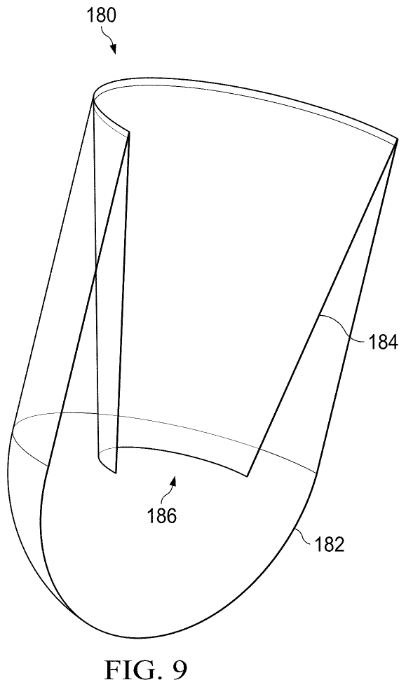

FIG. 9 is a perspective cross-sectional view of an implant delivery device 180 according to an embodiment. Similar to the embodiment of FIG. 1, the implant delivery device 180 includes of a sealed-end chamber (e.g., body portion) 182 and a delivery portion 184 (shown in an inverted configuration) with a single orifice 186 for insertion of the implant into the implant delivery device, and insertion of the implant into the patient. The sealed-end chamber 182 of FIG. 9 is curved. The curved shape may provide for better handling of the implant prior to insertion into the patient. Other shapes may also be used for the body portion, including the shapes shown in FIGS. 3A-F.

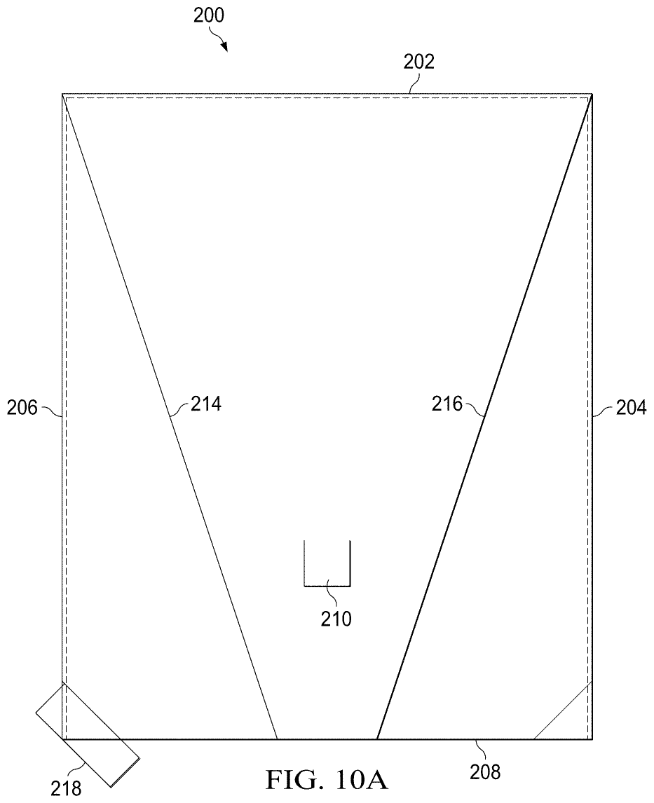

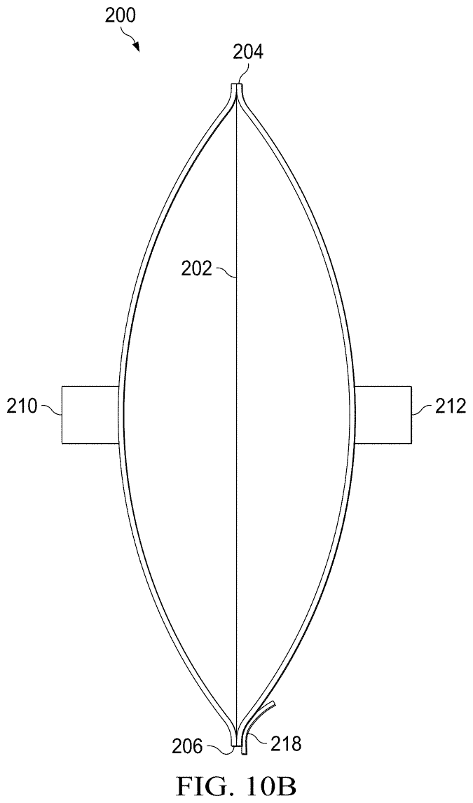

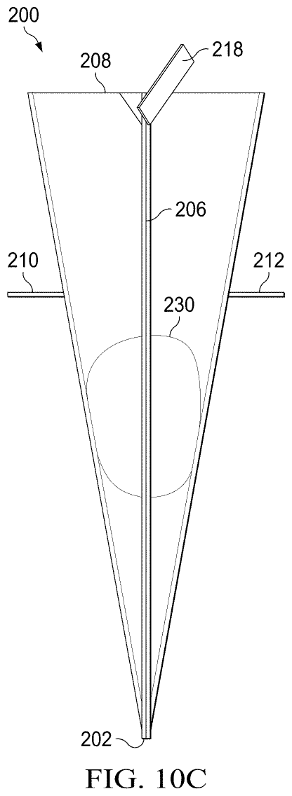

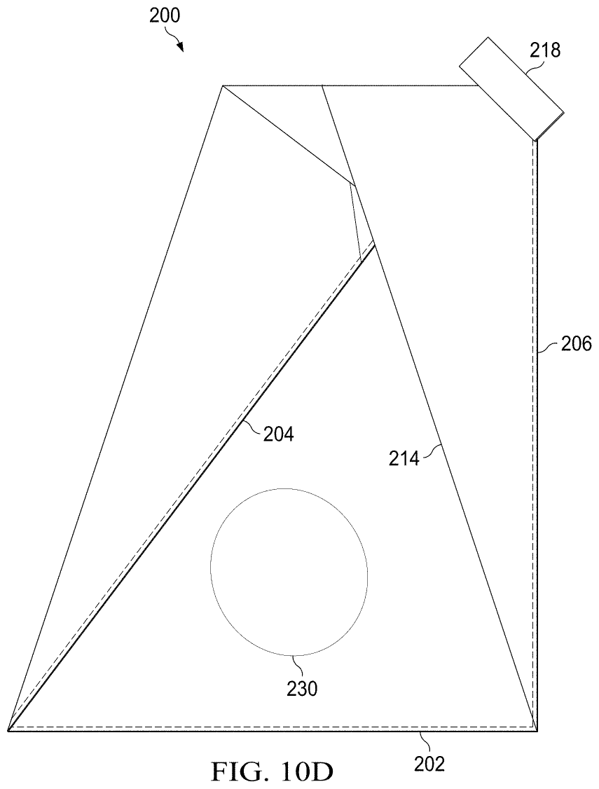

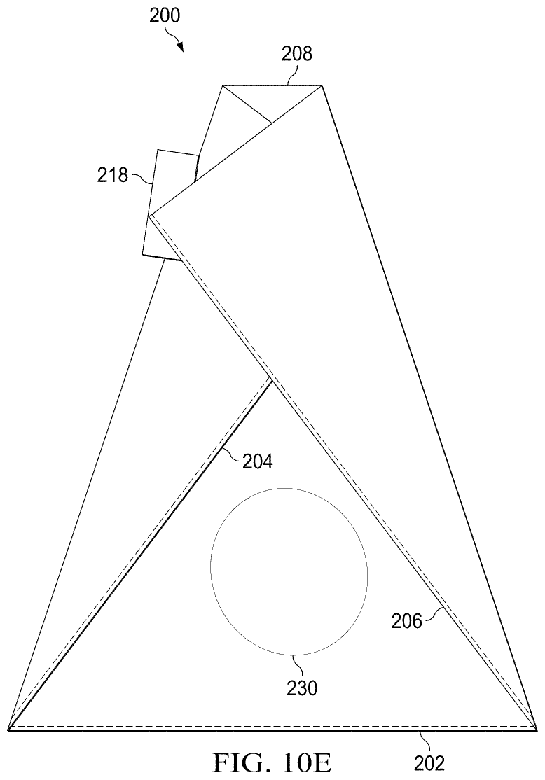

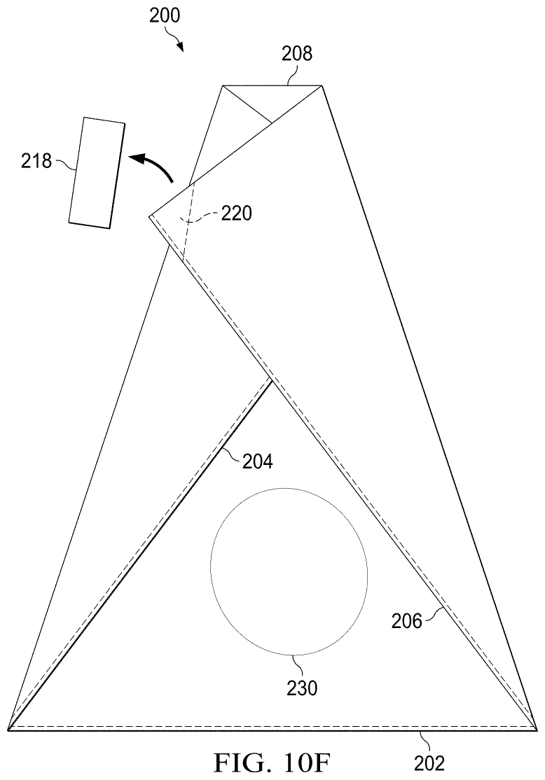

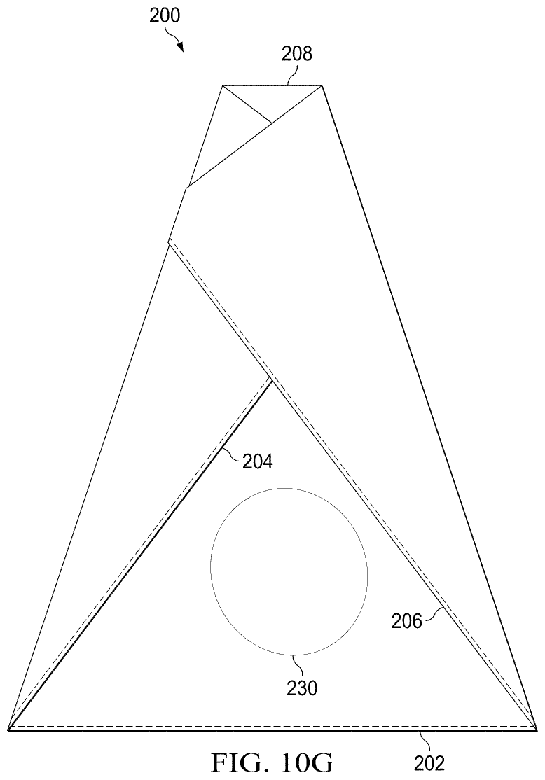

FIG. 10A illustrates a front view of an implant delivery device 200 in an unfolded configuration in accordance with an embodiment. The implant delivery device 200 includes a generally rectangular shape with a closed end 202, closed sides 204, 206 and an open end 208. The closed end 202 and closed sides 204 and 206 may be closed by a thermal weld, glue, tape, or other securement. In embodiments constructed from a lay flat tube, the closed sides 204 and 206 are provided by the lay flat tube itself and do not require further welding, gluing, etc. The lay flat tube is advantageous in that the material is continuous about a circumferential direction and the delivery device 200 can be manufactured by making one sealed seam to provide the closed end 202.

The implant delivery device 200 may include tabs 210 and 212 to facilitate opening the open end 208 (as shown in FIG. 10B). The tabs 210 and 212 may be respectively coupled to the faces or sides of the implant delivery device 200. The tabs 210 and 212 may also be formed as a part of and continuous with the substrate material of the implant delivery device. For example, the tabs 210 and 212 may be formed by slots cut into the face of the implant delivery device 200 thereby allowing them to be raised and manipulated.

The implant delivery device 200 may include indicia 214 and 216 for fold lines. In addition to or in place of the indicia, the implant delivery device may be creased along the lines 214 and 216. The indicia or creases may extend from one or both upper corners of the closed end 202 to the open end 208. The indicia or creases are angled such that at the open end 208, their width is less than a width of the open end 208.

FIG. 10B illustrates a top view of the implant delivery device 200 in an open configuration. The tabs 210 and 212 can be used in handling and opening of the implant delivery device 200 for an implant loading process. The tabs 210 and 212 may be secured to the sides of the implant delivery device 200 at respective ends of the tab 210 and 212 by glue or by passing through an opening or aperture in the side wall. The tabs 210 and 212 may also be continuous with the substrate and die cut as part of the shaping of the implant delivery device 200. A surgeon can tug on the tabs from one or both sides of the implant delivery device 200 to separate the sides to open the implant delivery device 200, and/or can push on one or more of the tabs to force the two sides of the implant delivery device together to at least partially close the implant delivery device 200.

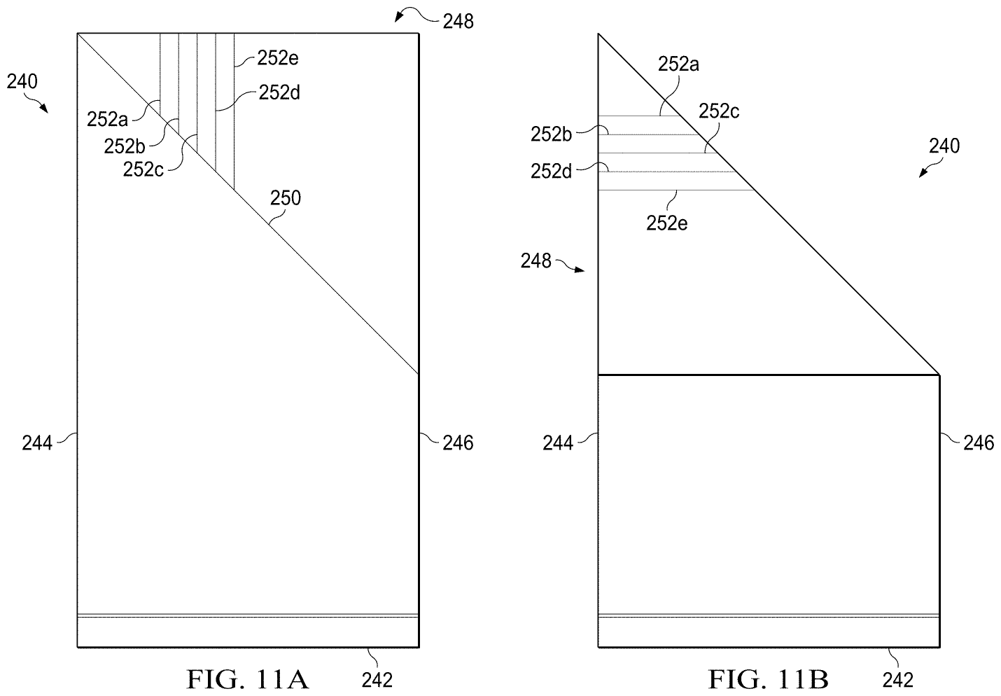

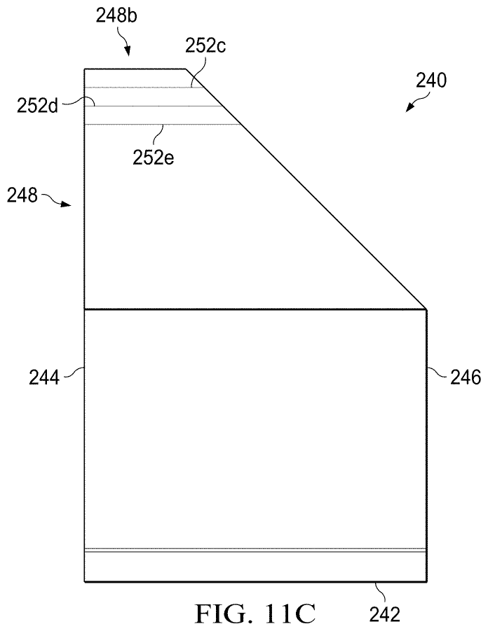

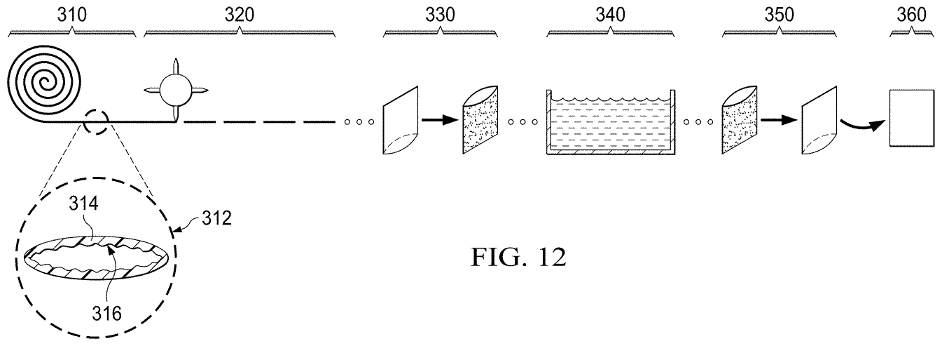

FIG. 10C, illustrates a side view of the implant delivery device 200 of FIG. 11B with an implant 230 disposed in the device 200. At this stage, the open end 208 is large having a width greater than a width of the implant 230, and the implant 230 may be easily disposed in the delivery device 200. The implant 230 may be passed through the opening formed by the surgeon separating the two sides of the implant delivery device 200 and positioned in the implant delivery device 203 (e.g., at the bottom or closed end 202 of the implant delivery device). Once the implant is positioned in the implant delivery device, the surgeon can add lubrication, if desired, to the implant and the inner surface of the implant delivery device. The surgeon may add the lubrication before or after closing the implant delivery device 200 as further described herein. Following the loading of the implant 230, the surgeon can proceed with closure of the implant delivery device.

FIG. 10D illustrates a front view of the implant delivery device 200 with a first side folded along the indicia or crease 214. In some embodiments, a first folded side may be a side without a tab (e.g., without the tab 218). A second folded side may be a side with a tab (e.g., with the tab 218). FIG. 10E illustrates a front view of the implant delivery device 200 in a folded configuration with two sides folded. The second side is folded along the indicia or crease 216. The tab 218 may be used to secure the sides to each other. FIG. 10F illustrates a front view of the implant delivery device 200 in a folded configuration in which the tab 218 has been removed, such as by peeling, to expose an adhesive region 220. The adhesive region 220 may be provided by single-sided or double sided adhesive tape. Clips, buttons, Velcro®, other adhesives, and the like may be used in addition to or in place of the adhesive region 220.

FIG. 10G illustrates a front view of the implant delivery device 200 in a folded configuration ready for the insertion of the open end 208 in the surgical site and the delivery (using, for example, the approach discussed above to encourage the implant forward and toward the open end 208) of the implant to the patient.

With reference to FIGS. 10A-10G, the indicia or creases 214 and 216 that provide guidance to the surgeon for making the folds may extend to the open end 208 of the implant delivery device proximal to a center line of the lower edge 208. In a preferred embodiment, a distance between the indicia or creases 214 and 216 at the open end 208 (of the indicia or creases 214 and 216 projected to the open end 208 of the indicia and creases do not extend to the end 208) is less than half of a width of the open end 208 in an unfolded configuration. The positioning of the indicia or creases as described provides an exit aperture formed by the closure of the two sides. It will be appreciated that the size of the aperture can be adjusted based on the positioning of the indicia and folds. The implant delivery device 200 may include multiple indicia 214, 216 corresponding to multiple implant sizes.

In some embodiments, at least a portion of the implant delivery device 200 includes a material that is at least partially transparent or translucent. The transparent or semi-transparent portions of the implant delivery device 200 may allow a surgeon to view the position of an enclosed implant and/or the position or motion of the implant during manipulation. This may be particularly advantageous in the case of a shaped implant to facilitate the delivery of the implant in the desired orientation. For example, at least a portion of the implant delivery device 200 may include transparent or translucent polymer film to allow a surgeon to view at least a portion of the implant.

In some embodiments, the implant delivery device 200 may be constructed of a flexible or semi flexible polymer or copolymer material. For example, the implant delivery device 200 may include one or more of polyethylene, polypropylene, polytetrafluoroethylene, polycarbonate, polyethylene terephthalate, fluoropolymer, ethylene vinyl acetate, polyvinylchloride, and silicon. The material may be provided as a film and bonded along a seam to provide the generically cylindrical shape. The material may also be provided by a lay flat tube that may limit or avoid the need for seams. As noted above, an advantage of a lay flat tube is that manufacturing steps for bonding a vertical seam may be limited or avoided thereby increase yields and decreasing manufacturing costs. The lay flat tube may also limit or reduce the number of seams thereby reducing possible weak points and increasing the strength of the device 200. Preferably, the device 200 will not fail or yield under forces that may be exerted on the device by a surgeon.

In some embodiments, one or more of the internal surfaces of the implant delivery device 200 may be fabricated or treated to provide a low friction coefficient. This may be achieved through the use of a lubricious coating. A lubricious coating or surface may include a coating or surface of an object having slipperiness, low friction and/or smoothness. This description is exemplary in nature and a lubricious coating or surface is not defined by or limited thereto. For example, in some embodiments, at least a portion of at least one or more of the internal surfaces of the implant delivery device 200 can be lubricious. The lubricity may be achieved with or without a coating on the material of the implant delivery device 200. The lubricity may be provided or enhanced by a hydrophilic coating or a hydrophobic coating. In some embodiments, the lubricity includes a combination of the lubricious nature of the material (for example forming the walls) of the implant delivery device 200, and a coating on the material of the implant delivery device 200.

The size and/or shape of the implant delivery device 200 may be different based on surgeon requirements and/or patient requirements. For example, the open end 208 may have different sizes and dimensions, and the sides 204, 206 may be straight or angled.

FIG. 11A illustrates a front view of an implant delivery device 240 in an unfolded configuration in accordance with an embodiment. Similar to the implant delivery device 200 of FIGS. 10, the implant delivery device 240 includes a generally rectangular shape with a closed end 242, closed sides 244, 246 and an open end 248. The closed end 242 and closed sides 244 and 246 may be closed by a thermal weld, glue, tape, or other securement. In embodiments constructed from a lay flat tube, the closed sides 244 and 246 are provided by the lay flat tube itself and do not require further welding, gluing, etc. The lay flat tube is advantageous in that the material is continuous about a circumferential direction and the delivery device 240 can be manufactured by making one sealed seam to provide the closed end 242.

The implant delivery device 240 includes indicia 250 for a fold line. The indicia 250 preferably extends from a corner of the open end 248 diagonally, for example at 45 degrees, to an opposite side of the delivery device 240. In addition to or in place of the indicia 250, the implant delivery device may be creased along the lines 250.

The implant delivery device 240 may also include one or more indicia 252a-252e extending from the indicia 250 to the open end 248. In the flat configuration, the indicia 252a-252e are approximately vertical. The indicia 252a-252e may also be angled to approximate an isosceles triangle for symmetry at the insertion end after folding.

FIG. 11B illustrates a front view of the implant delivery device 240 with a side folded along the indicia or crease 250. When folded, the open end 248 is directed to the side and the indicia 252 become oriented more horizontally. Referring to FIG. 11C, in the folded configuration, a portion may be cut away or otherwise removed along one of the indicia 252a-252e. The removal of the corner at the indicia actually enlarges the open end 248 to extend down the side 246. However, because of the fold, the cross-sectional area available for the implant to pass through is reduced.

Referring to FIG. 12, an exemplary process for manufacturing an implant delivery device according to any of the embodiments described above will be described. At 310, a roll of bulk material is provided. The bulk material may be a biocompatible film such as the thermoplastic films discussed above. The bulk material may be a roll of lay flat tubing. Outset 312 illustrates an exemplary cross-section of lay flat tubing. The lay flat tubing 314 is continuous about a circumference and, in some embodiments, includes a textured surface 316 on an inner portion of the lay flat tubing 314. Exemplary advantages of the textured surface include reducing a sticking effect of the walls of the lay flat tubing 314 from sticking together (such as by static, etc), improving the ability to open and invert the tubing as discussed further below, and facilitating the application of a treatment to the lay flat tubing.

At 320, the bulk material is shaped into shape of the implant delivery device. In the case of lay flat tubing, a rotating hot knife apparatus may both cut the bulk lay flat tubing 314 to size as well as perform thermal welding of one end of the implant delivery device to close one end and leave one end open. A die cutting operation may be used in place of or addition to a rotating hot knife apparatus. In some embodiments, the end of the implant delivery device is not bonded until later in the process such as after the re-inversion. If film sheets are used for bulk material or other shaping such as applying angled seams, securing layers of film sheets and other shaping may be performed in this step.

At 330, the cut and shaped material is turned inside out. In the case of lay flat tubing, the ridges 316 may now be exposed on an exterior.

At 340, the inverted material may be dipped in a treatment. The treatment may be, for example, a lubricious coating as discussed above. Applying the lubricious coating may include preparing a liquid form of the coating if the coating is not already liquid. The coating material may be dissolved in a solvent such as alcohol or water. After dipping, the coating may be cured by drying, heating, exposure to ultra violet light, and the like. Once cured, the coating may be covalently bonded to the bulk material of the device such that subsequent exposure to liquid does not fully remove the coating from the bulk material. For example, the coating may include Serene ® available from Surmodics, ComfortCoat® available from DSM, ISurGlide® available from ISurTec®, Lubricent® available from Harland Medical Systems, Hydak® available from Biocoat and parylene. It will be appreciated that coatings applied with or without solvents and other delivery methods to apply the coating may be used. For example, the coating may be sprayed on, rolled on, poured on, and so forth. The coating may also be applied selectively, for example by masking off areas of the device prior to dipping, stamping on the coating material, using a slot die application process, roll coating, or applying the coating material through an aperture screen. Examples of patterns include dots, a checkerboard pattern and vertical stripes extending from the open end to the closed end of the device. The coating may also be selectively applied to regions of the device, for example starting from proximal orifice end and extending partially but not completely to an opposite end of the device. The selective application of the coating material may reduce the cost of the device while still reducing the coefficient of friction to provide desirable performance.

Once the coating is cured, at 350, the devices may be inverted again to be placed back in a not inverted configuration. At this step, a shipping protection sheet may optionally be added inside the device to limit contact and sticking between the coated interior surfaces of the device. At 360, the devices may be packaged for shipment. In some embodiments, this includes disposing an implant inside the device and sterilizing the device.

It will be appreciated that in some embodiments, the inversion of the material may be performed before the shaping and coating. For example, the section of bulk material may be inverted and both ends of the section of material (e.g., the lay flat tube), be sealed to prevent entry of the lubricious material. After dip coating, one or both seals may be cut off, the material re-inverted, and then sealed (and in some embodiments shaped). Portions of the inverted material may be masked off before dip coating or the coating may be applied by a selective process such as spraying to limit the application of coating material where seals will be formed to improve the strength of the seal at those locations.

It will also be appreciated that in place of or in addition to a lubricious coating, other treatments may also be provided. For example, an antimicrobial treatment may be applied to one or more surfaces of the implant delivery device.

It will be appreciated by those skilled in the art that while particular embodiments and examples, have been described above, the disclosure is not necessarily so limited, and that numerous other embodiments, examples, uses, modifications and departures from the embodiments, examples and uses are intended to be encompassed by the description and figures and the appended claims. Accordingly, the specification and figures are to be regarded in an illustrative rather than a restrictive sense, and all such modifications are intended to be included within the scope of the present disclosure. Any benefits, advantages, or solutions to problems that are described herein with regard to specific embodiments are not intended to be construed as a critical, required, or essential feature or element of any or all the claims.

The title, abstract, background, and headings are provided in compliance with regulations and/or for the convenience of the reader. They include no admissions as to the scope and content of prior art and no limitations applicable to all disclosed embodiments.

1. An implant delivery device, comprising:
a body portion having a first sidewall, a second sidewall, and a closed end; and
an implant delivery portion disposed opposite to the closed end, the implant delivery portion include a first sidewall, a second sidewall, and an orifice, the first sidewall is angled with respect to the first sidewall of the body portion, and the second sidewall is angled with respect to the second sidewall of the body portion.

2. The implant delivery device of claim 1, wherein the closed end includes an end wall bonded to at least one face of the body portion.

3. The implant delivery device of claim 1, wherein the closed end includes a bond between at least one face of the body portion.

4. The implant delivery device of claim 3, wherein the body portion includes a lay flat tube, and the thermal bond includes a bond closing the lay flat tube.

5. The implant delivery device of claim 1, wherein the implant delivery portion is configured to invert such that the orifice is disposed inside the body portion.

6. The implant delivery device of claim 5, wherein the implant delivery portion is configured to receive an implant while in an inverted configuration such that the implant is disposed inside the body portion.

7. The implant delivery device of claim 6, wherein
the implant delivery portion is configured to return to a not inverted configuration while an implant is disposed in the body portion, and
in the not inverted configuration, the orifice is not disposed inside the body portion.

8. The implant delivery device of claim 1, wherein an interior surface of the implant delivery portion includes a friction reducing coating.

9. The implant delivery device of claim 8, wherein the friction reducing coating is hydrophilic.

10. The implant delivery device of claim 1, wherein an interior surface of the implant delivery portion includes an antimicrobial treatment.

11. The implant delivery device of claim 1, wherein the orifice is an only opening of the implant delivery device.

12. An implant delivery device, comprising:
a first closed sidewall;
a second closed sidewall disposed opposite the first closed sidewall;
a third closed sidewall disposed between the first closed sidewall and the second closed sidewall; and
an open end disposed opposite the third closed sidewall and disposed between the first closed sidewall and the second closed sidewall, wherein
a face of the implant delivery device disposed between the first closed sidewall and the second closed sidewall is configured to fold such that a cross-sectional area of the open end is reduced.

13. The implant deliver device of claim 12, wherein
the face of the implant delivery device includes an indicia, and
the indicia extends from proximal a corner of the first closed sidewall and the open end toward the second closed sidewall.

14. The implant delivery device of claim 13, wherein the indicia includes a crease.

15. The implant delivery device of claim 13, wherein the face includes a protruding tab.

16. The implant delivery device of claim 13, wherein the indicia extends at approximately a 45 degree angle with respect to the open end.

17. The implant delivery device of claim 13, further comprising a second indicia extending from the first indicia towards the open end.

18. A method of manufacturing an implant delivery device, comprising:
providing a section of lay flat tubing;
inverting the lay flat tubing to arrange the lay flat tubing in an inverted configuration;
applying a friction reducing treatment to the inverted lay flat tubing;
inverting the lay flat tubing to arrange the lay flat tubing in a not inverted configuration; and
sealing an end of the section of lay flat tubing.

19. The method of claim 18, wherein the lay flat tubing includes a thermoplastic material, and sealing the end of the section of lay flat tubing includes thermally bonding the thermoplastic material.

20. The method of claim 18, wherein in a not inverted configuration, an interior surface of the lay flat tubing is textured.

21. The method of claim 18, wherein the applying the friction reducing treatment includes dipping the inverted lay flat tubing in a lubricious material.

22. The method of claim 21, further comprising curing the lubricious material before inverting the lay flat tubing to arrange the lay flat tubing in a not inverted configuration.

23. The method of claim 21, wherein the lubricious material is hydrophilic.

24. A method of inserting an implant, comprising:
providing an implant delivery device, the device including:
a first closed sidewall,
a second closed sidewall disposed opposite the first closed sidewall,
a third closed sidewall disposed between the first closed sidewall and the second closed sidewall, and
an open end disposed opposite the third closed sidewall and disposed between the first closed sidewall and the second closed sidewall;
inserting the implant into the implant delivery device through the open end;
folding the first closed sidewall towards the second closed sidewall to reduce a cross sectional area of an orifice provided by the open end; and
applying a force to the implant delivery device to cause the implant to move towards and through the orifice.

25. The method of claim 24, further comprising folding the second closed sidewall towards the first closed sidewall to reduce a cross sectional area of the orifice.

26. The method of claim 24, further comprising removing a corner formed by the open end and the second closed side wall.

27. The method of claim 26, wherein the removing includes cutting.

28. The method of claim 26, wherein an interior surface of the implant delivery device includes a friction reducing treatment.

29. The method of claim 28, wherein the friction reducing treatment includes a hydrophilic coating.

30. The method of claim 29, further comprising hydrating the friction reducing treatment.
